(19) United States
(12) Patent Application Publication
(10) Pub. No.: US 2006/0173468 A1
(43) Pub. Date: Aug. 3, 2006

(54) Obturator introducer with snare

(76) Inventors: Marc Simmon, Becker, MN (US); Martin T. Steele SR., Otsego, MN (US); Emily Daley, Brooklyn Center, MN (US); Dennis Femrite, Andover, MN (US)

Correspondence Address: FISH & RICHARDSON P.C., PO BOX 1022, MINNEAPOLIS, MN, 55440-1022, US

(21) Appl. No.: 11/046,392

(22) Filed: Jan. 28, 2005

(51) Int. Cl. A61B 17/26

(52) U.S. Cl. 606/113

(57) ABSTRACT

Devices for introducing and positioning urethral slings within female subjects are described herein. Also described are methods for using such devices, as well as articles of manufacture containing the devices.

### TECHNICAL FIELD

This invention relates to devices for introducing and/or positioning medical objects in a subject, and more particularly to devices for introducing a transobturator into a subject.

### BACKGROUND

One method for treating urinary incontinence in women involves the use of implantable urethral slings (also referred to herein as transobturators). A urethral sling can be, for example, a tape made of a biocompatible material such as meshed, woven, or knitted polypropylene or polyethylene. Such a device can be positioned under the urethra and extended such that the free ends of the device are pulled into the region of the two obturator foramina of the iliac wing, where they can be anchored into the abdominal wall. A urethral sling such as the Mentor ObTape typically is positioned by inserting a curved needle (e.g., an Emmet needle) into the groin in the region of an obturator foramen, advancing the needle through the groin and into the vagina through an incision therein, feeding one end of the sling through the eye of the needle, retracting the needle out of the body from its point of insertion, and repeating the procedure for the opposite side of the body and the second end of the sling. Such a procedure is described in, for example, U.S. Pat. No. 6,638,211.

### SUMMARY

This document discloses devices for introducing and positioning transobturators within female subjects. These introducer devices may feature an elongate curved member having a snare disposed therein. The snare, which can be formed as a loop or can have a loop at its distal end, can be extended from and retracted into the elongate curved member. Because the loop typically is larger than the eye of the Emmet needle used in previously described procedures, threading of a transobturator tape into the introducer can be facilitated. In addition, the ability to extend the snare from the elongate curved member can effectively increase the reach of the introducer, which can better accommodate larger subjects. The ability to retract the snare into the elongate curved member once the end of the transobturator tape has been passed through the loop can lock the tape into the introducer, essentially eliminating or at least greatly reducing the possibility of separation during passage through the subject's tissues during positioning of the tape. Further, use of the introducer devices described herein can provide for a smoother transition between the introducer and the transobturator material than can be achieved using an Emmet needle, for example.

In one aspect, the invention features a device having: (a) a handle, (b) an elongate curved member defining a lumen, the member having a proximal end attached to the handle and an open distal end, and (c) a snare slidably disposed proximate to the distal end of the lumen. The snare can have a loop. The device can further include a flexible elongate member disposed in the lumen and attached to the loop to move the loop longitudinally in or out of the open distal end. The loop can expand when extended outside the elongate curved member. The loop can be made of a braided material. The snare can have a pair of interengaging jaws. The snare can contain metal (e.g., nitinol). The snare can have a coating (e.g., a coating containing polyether ether ketone or teflon) over the metal. The snare can close on a material passed through the snare when the snare is retracted at least in part into the open distal end of the elongate curved member.

The elongate curved member can be shaped to be guided in a predetermined path for extending from the vicinity of an obturator foramen in a female subject to the vicinity of the vagina of the subject. The predetermined path can include, from the proximal end toward the distal open end, a first straight segment, a first convex curved segment, a second straight segment, and a second concave curved segment. The predetermined path can be shaped for positioning a urethral sling between the urethra of a female subject and an obturator foramen of the subject. The distal open end of the elongate curved member can have a beveled opening or a flat opening.

The device can further include an actuator associated with the handle for manual operation by a user, wherein the actuator is connected to the snare so that movement of the actuator causes extension or retraction of the snare. The actuator can be slidable or pivoting. The actuator can be rotatable and thereby move linearly relative to the handle to extend or retract the snare. The actuator can have one or more protrusions to receive force from a user's hand. The actuator can have a top surface and a bottom surface, wherein the top and bottom surfaces define protrusions to receive force from a user's hand. The actuator can have an slot through which a pin can be passed from a first side of the handle to a second side of the handle.

In another aspect, the invention features a method for introducing a transobturator into a female subject. The method can include:

(a) guiding an introducer having a retractable snare through a perineal incision opposite an obturator foramen on a first side of the subject, through the abdomen of the subject, and through a vaginal wall of the subject;

(b) extending the snare from the introducer;

(c) inserting a transobturator into the vagina of the subject and passing a first end of the transobturator through the snare;

(d) retracting the snare to hold the first end of the transobturator; and

(e) withdrawing the transobturator to an area proximate the perineal incision.

The method can further include making an incision in the vaginal wall. The method can further include withdrawing the first end of the transobturator through the perineal incision. The method can further include, subsequent to step (e), cutting off an excess portion of the transobturator flush with the skin. The method can further include repeating steps (a) through (e) on a second side of the subject so that a second end of the transobturator is positioned proximate a perineal incision opposite a second obturator foramen of the subject.

In another aspect, the invention features a kit containing a transobturator and one or more transobturator introducers, each of the transobturator introducers having a handle, an elongate curved member defining a lumen and having an open distal end and a proximal end attached to the handle, and a snare slidably disposed in the lumen proximate the open distal end. The kit can contain two the transobturator introducers.

In yet another embodiment, the invention features a device having: (a) a handle, (b) an elongate curved member defining a lumen, the member having a proximal end attached to the handle and a distal end, and (c) a means for holding a transobturator near the distal end of the elongate curved member.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a transobturator introducer in which the snare is in an extended position.

FIG. 2 is a perspective view of a transobturator introducer in which the snare is in a retracted position.

FIG. 3 is a perspective view showing the individual components of a transobturator introducer.

FIGS. 4A and 4B are close-up views of the distal end of a snare having an alternate configuration for the loop.

FIG. 5 is a close-up view of the distal end of a snare having interengagable jaws.

FIG. 6 is a close-up view of a rotary actuator.

FIG. 7A is cross-sectional end view of a handle having top and bottom inner surfaces that are slanted. FIG. 7B is an overhead view of a handle having angled inner side surfaces.

Like reference symbols in the various drawings indicate like elements.

### DETAILED DESCRIPTION

The transobturator introducers and the methods provided herein can be used to introduce and position urethral slings within female subjects. The introducer devices can facilitate the placement of urethral slings by decreasing the time required for positioning of the slings and by reducing the potential for damage to the tissues of subjects into which the slings are implanted.

Devices

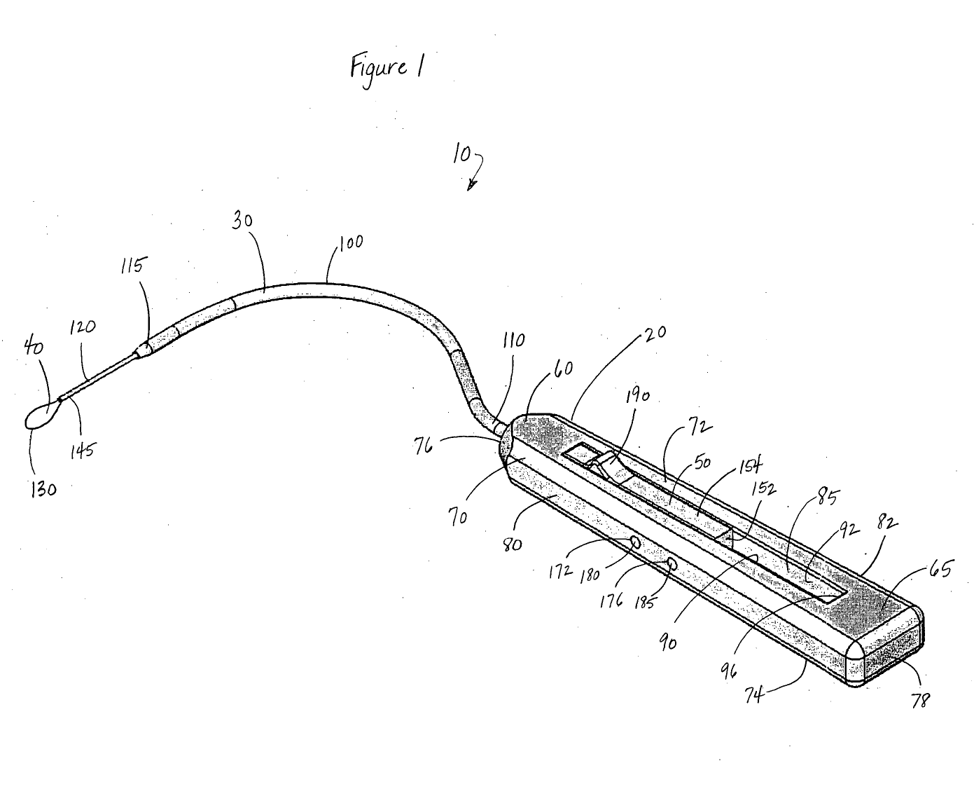

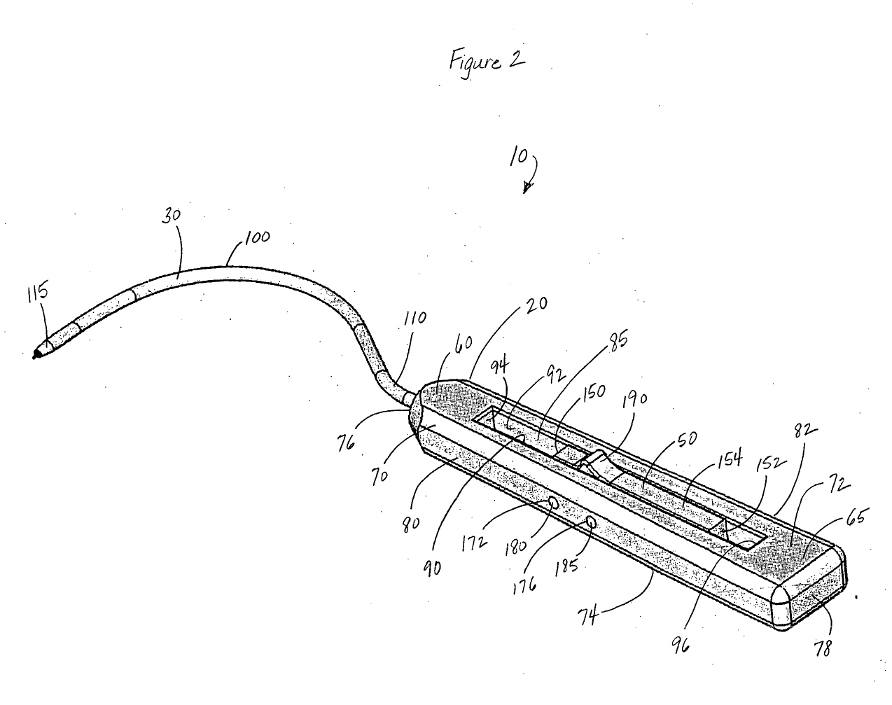

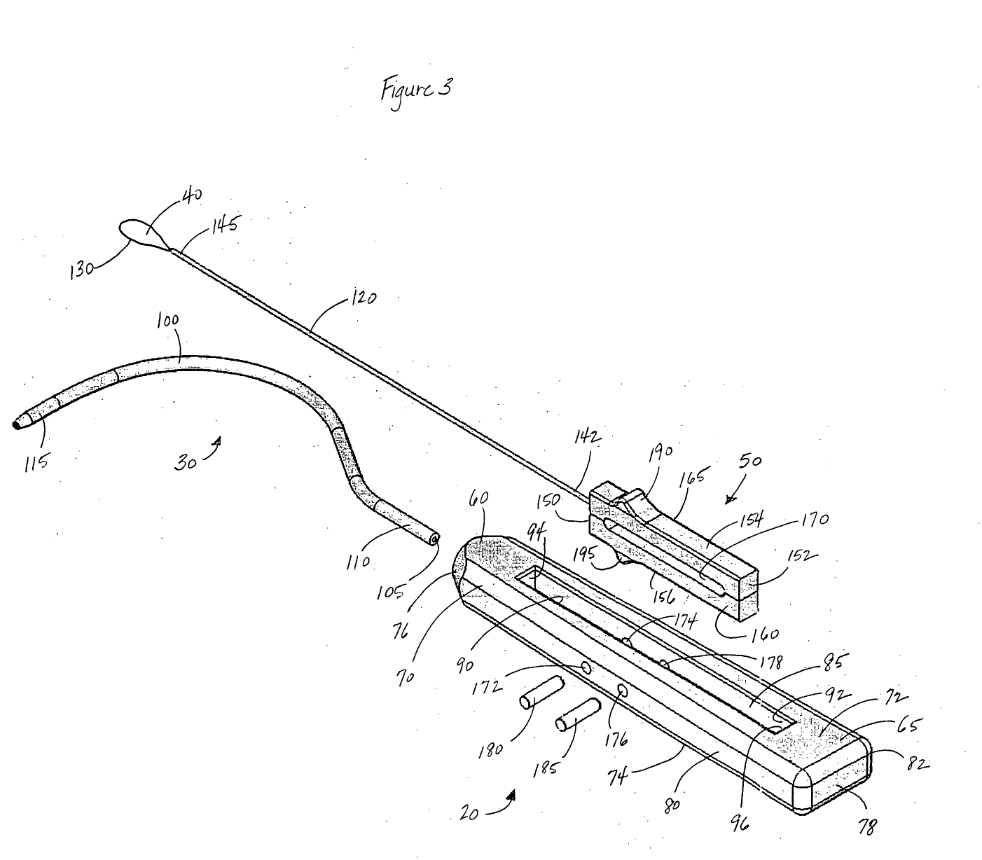

FIGS. 1 and 2 are perspective views of a transobturator introducer in which the snare is in an extended position or a retracted position, respectively. FIG. 3 is a perspective view showing the individual components of a transobturator introducer. Introducer 10 generally includes three major components: an elongate curved member that can be inserted into a subject's body to extend between, for example, an area opposite an obturator foramen and the vicinity of the subject's vagina, a snare that can be extended from a distal end of the elongate curved member so that a user (e.g., a clinician) can grasp and retain a portion of a transobturator, and a handle at the proximal end of the elongate curved member. The handle can have an actuator that, when operated, extends or retracts the snare.

A user can grasp handle 20 to manipulate introducer 10. Handle 20 can have any suitable configuration. For example, handle 20 can have first end 60 and second end 65. Handle 20 also can have outer surface 70, which can include, for example, top outer surface 72, bottom outer surface 74, first outer end surface 76, second outer end surface 78, first outer side surface 80, and second outer side surface 82. Handle 20 also can have a hollow or open portion. For example, open portion 85 of handle 20 can be defined by first inner side surface 90, second inner side surface 92, first inner end surface 94, and second inner end surface 96. As described below, open portion 85 can be configured to contain actuator 50. In addition, first end 60 of handle 20 can define channel 98 (not visible in FIGS. 1-3), which can extend between first outer end surface 76 and first inner end surface 94.

Handle 20 can be made using any suitable method, including methods that are standard in the art. For example, handle 20 can be molded or machined in a single piece or in multiple pieces. In one embodiment, handle 20 can be molded or machined as two separate pieces that can be assembled and held together using, for example, a snap fit, a friction fit, a connecting device such as a bolt, screw, or clamp, or can be joined by an adhesive or weld, such as sonic welding. Handle 20 also can be made from any suitable material. For example, handle 20 can contain plastic [e.g., a moldable or machinable thermoplastic such as polycarbonate, polypropylene, polyethylene, polystyrene, or polyvinyl chloride, or a thermoplastic copolymer containing, for example, polycarbonate and acrylonitrile butadiene styrene (ABS)]. Alternatively or in addition, handle 20 can contain metal (e.g., stainless steel) or any other suitable material.

Elongate curved member 30 can be shaped to be inserted into the female body opposite an obturator foramen and guided through the groin to the vicinity of the vagina. Elongate curved member 30 can have outer surface 100, lumen 105 (visible only in FIG. 3), proximal end 110, and distal end 115. Proximal end 110 of elongate curved member 30 can be attached to handle 20 at first end 60 such that, through channel 98, lumen 105 can be in fluid communication with open portion 85 of handle 20. Distal end 115 can be open. In some embodiments, distal end 115 can have a beveled opening. In such embodiments, elongate curved member 30 can be a curved needle. In other embodiments, distal end 115 can have a flat opening.

Elongate curved member 30 can be rigid or malleable, and can have any suitable shape. In some embodiments, elongate curved member 30 can have a predetermined shape. For example, elongate curved member 30 can have one or more curved portions and/or one or more portions that are essentially straight. As depicted in FIGS. 1-3, elongate curved member 30 can have, from proximal end 110 toward distal end 115, a first straight portion, a convex curved portion, a second straight portion, and a concave curved portion. The distance along the length of elongate curved member 30 can be, for example, between about 15 cm and about 30 cm (e.g., between about 20 cm and about 28 cm, between about 22 mm and about 26 cm, or between about 23 cm and about 25 cm).

Elongate curved member 30 also can be made from any suitable material. For example, elongate curved member 30 can contain metal, plastic, or any combination thereof. In some embodiments, elongate curved member 30 can contain stainless steel. Alternatively, elongate curved member 30 can be an extruded plastic material that is formed into shape either immediately upon being extruded or at a later time, such as by heating the materials and then shaping it.

Proximal end 110 can be attached to handle 20 using any suitable apparatuses, including, without limitation, a snap fit, a friction fit, a threaded fit, a connector such as a bolt, screw, or clamp, or an adhesive. In some embodiments, proximal end 110 of elongate curved member 30 can be split or splined, and can be configured to snap into a handle constructed in two parts (e.g., a handle constructed in a top part and a bottom part).

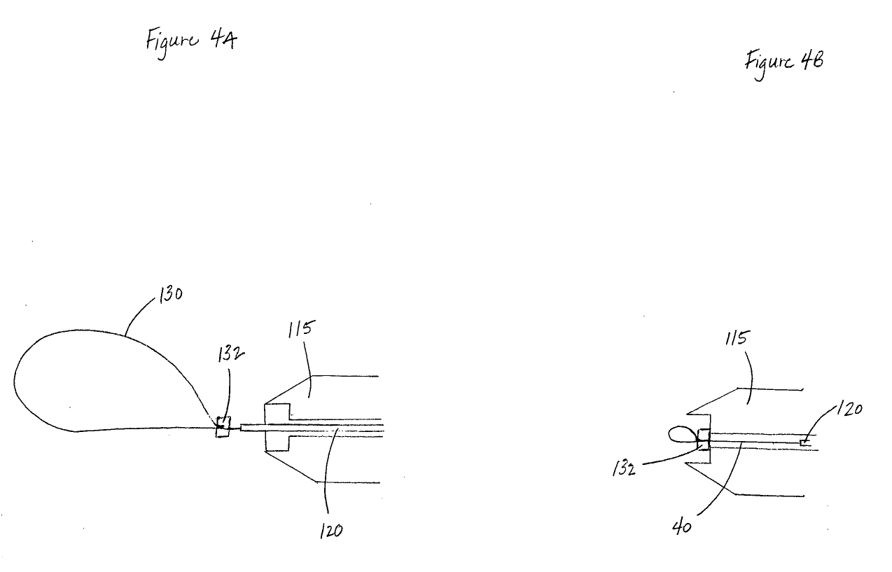

Snare 40 can be positioned in distal end 115 of elongate curved member 30. Snare 40 can be attached to rod 120, which can slide longitudinally in lumen 105 of elongate curved member 30. Snare 40 may take the form of a fixed loop (e.g., loop 130) that slides in an out of distal end 115 of elongate curved member 30. An item such as a transobturator tape that is passed through loop 130 can be held against or within distal end 115 of elongate curved member 30 when loop 130 is retracted. Snare 40 also may be in the form of a sliding loop, such as one in which the loop is formed of a flexible cord or wire having a passage at one end through which the remainder of the cord or wire passes, much like a lasso. FIGS. 4A and 4B show in more detail a sliding loop snare. In this embodiment, snare 40 can have sleeve 132 fixedly attached to its distal end. Loop 130 can be formed by a doubling back of snare 40 through sleeve 132, such that the size of loop 130 is increased (FIG. 4A) as rod 120 is advanced toward distal end 115 of lumen 105. Conversely, the size of loop 130 can be decreased (FIG. 4B) as rod 120 is retracted into lumen 105. As the size of snare 130 decreases, a transobturator device passed through loop 130 can become tightly retained therein. Sleeve 132 may be held loosely in open distal end 115 of elongate curved member 30, as shown, to permit further extension of loop 130. Alternatively, sleeve 132 may be fixed at open distal end 115 of elongate curved member 30.

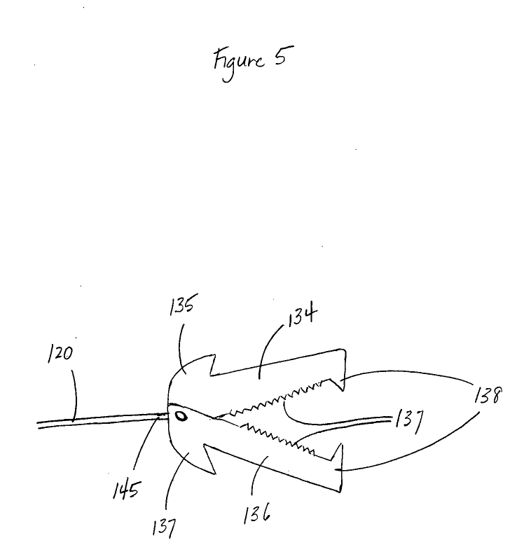

Snare 40 also could take the form of a pair of interengageable grasping jaws (FIG. 5). The jaws could pivot at the distal end of rod 120 and may be biased into an open position, such as by a spring. The jaws then may be forced closed when they are drawn or retracted into distal end 115 of elongate curved member 30 by contact of their back surfaces with distal end 115. Each jaw may have a knurled or similar surface to better grasp an object placed between the jaws. An embodiment in which the snare is comprised of a pair of jaws is shown in FIG. 5. Jaws 134 and 136 can define shoulders 135 and 137, respectively, which can allow jaws 134 and 136 to open when snare 40 is extended, but which cause jaws 134 and 136 to close when snare 40 is retracted. Further, jaws 134 and 136 can define ridges 137 and/or teeth 138 to facilitate gripping of a transobturator device during use.

Where snare 40 is in the form of loop 130, loop 130 can define an opening of any adequate size when snare 40 is extended from device 10. In some embodiments, when loop 130 is in a open position, its length can be greater than its width. For example, loop 130 can have a length between about 1.5 cm and about 4.25 cm (e.g., between about 2 cm and about 3.75 cm, between about 2.25 cm and about 3.5 cm, or between about 2.5 cm and about 3.25 cm). Loop 130 also can have a width between about 1.4 cm and about 2.4 cm (e.g., between about 1.65 cm and about 2.15 cm, between about 1.8 cm and about 2.0 cm, or about 1.9 cm). In general, loop 130 can be sized to open into a cross-section that is substantially larger than the opening at distal end 115 of elongate curved member 30. In this manner, a user (e.g., a clinician) can more easily insert a portion of a transobturator tape into snare 40.

Loop 130 can be made of a flexible and resilient material that can be compressed side-to-side when retracted into distal end 115 of elongate curved member 30. Loop 130 then may expand from a flattened shape inside lumen 105 to a more circular or teardrop shape to facilitate threading of a transobturator through loop 130. Loop 130 can contain, for example, metal (e.g., stainless steel), an alloy (e.g., nitinol), or plastic. Heat-treated nitinol may be particularly useful. In some embodiments, loop 130 can be made from a braided material. In addition, loop 130 can be coated with or contained within a material to provide lubricity and resistance to abrasion. For example, loop 130 can be coated with or contained within a sleeve of a material such as polyether ether ketone (PEEK) or teflon.

Rod 120 can have proximal end 142 and distal end 145. Proximal end 142 can extend through proximal end 110 of elongate curved member 30, through channel 98 of handle 20, and into open portion 85 of handle 20, where, as described below, it can be fixedly attached to actuator 50. Distal end 145 can be positioned near distal end 115 of elongate curved member 30, such that it is contained within elongate curved member 30 when in a retracted position and is extended to or beyond distal end 115 of elongate curved member 30 when in an extended position.

Rod 120 can have any suitable size. Typically, the length of rod 120 is similar to or slightly greater than the length of elongate curved member 30. In some embodiments, rod 120 can have a length between about 15 cm and about 35 cm (e.g., between about 18 cm and about 33 cm, between about 21 cm and about 31 cm, or between about 24 cm and about 28 cm).

Rod 120 can be formed of a flexible material so that it can follow the shape of elongate curved member 30 as it is moved longitudinally within lumen 105. For example, rod 120 can be a wire containing metal (e.g., stainless steel), an alloy (e.g., nitinol) or plastic. Heat-treated nitinol may be particularly useful. In some embodiments, rod 120 can be coated with or contained within a sleeve of PEEK or teflon.

Handle 20 may be provided with actuator 50 to allow for extension and retraction of snare 40. Actuator 50 can have any suitable shape that allows for convenient operation of introducer 10. As depicted in FIGS. 1-3, for example, actuator 50 can have first end 150, second end 152, top surface 154, bottom surface 156, first side surface 160, and second side surface 165. Actuator 50 can have any suitable size. In the embodiments shown in FIGS. 1-3, actuator 50 can be approximately the same width as open portion 85, but can have a length less than that of open portion 85 so that actuator 50 can move longitudinally within open portion 85.

First end 150 of actuator 50 can be attached to proximal end 142 of rod 120. The attachment can be in any suitable form (e.g., a snap fit, a threaded fit, a friction fit, using attachment means such as a screw, a bolt, or a clamp, or joining with an adhesive or weld). In one embodiment, for example, rod 120 can have a coating (e.g., a PEEK or teflon coating) that extends past proximal end 142 and that engages a pin or other protrusion defined by first end 150 of actuator 50.

Actuator 50 can be mounted to handle 20 using any suitable means. As shown in FIG. 3, for example, first and second side surfaces 160 and 165 of actuator 50 can define slot 170 extending therebetween, while handle 20 define apertures 172, 174, 176, and 178 extending between first and second outer side surfaces 80 and 82 and first and second inner side surfaces 90 and 92. Apertures 172, 174, 176, and 178 can be adapted to retain pins 180 and 185. In some embodiments, one end of pin 180 can be retained in aperture 172, the midsection of pin 180 can extend through slot 170, and the other end of pin 180 can be retained in aperture 176. Similarly, one end of pin 185 can be retained in aperture 174, the midsection of pin 185 can extend through slot 170, and the other end of pin 185 can be retained in aperture 178. In these embodiments, slot 170 may have a width slightly larger than the diameter of pins 180 and 185. In this manner, pins 180 and 185 can retain actuator 50 within open portion 85, while permitting actuator 50 to be slidably movable within handle 20.

Any other suitable mounting structure that permits suitable motion of actuator 50 also can be used. In another embodiment, for example, side surfaces 160 and 165 of actuator 50 each can define a groove that can be engaged by pins or other protrusions extending from first and second inner side surfaces 90 and 92 or handle 20 into open portion 85. As above, the interaction between such grooves and protrusions can be secure enough to retain actuator 50 within open portion 85, but can have enough play to allow actuator 50 to move within open portion 85. In still another embodiment, actuator 50 can be mounted to one or more outer surfaces of handle 20, but can contain a portion that extends into open portion 85 for attachment to rod 120.

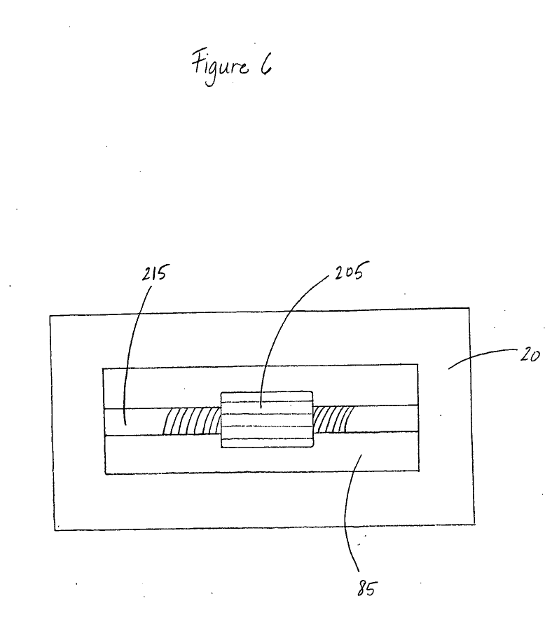

Introducer 10 can include any structure by which a user can move actuator 50 within or upon handle 20. If actuator 50 is slidably movable, for example, actuator 50 can define fins, ridges, indentations, or any other features by which a user could achieve movement of actuator 50. As shown in FIGS. 1-3, actuator 50 can have protrusions 190 and 195 that a user can engage with their fingers to slide actuator 50 either toward or away from first end 60. In another embodiment depicted in FIG. 6, for example, actuator 200 can be rotatable, such that when it is rotated it moves longitudinally with respect to handle 20. In this embodiment, actuator 200 can include knob 205, which can be internally threaded, and externally threaded shaft 215. Knob 205 can be knurled or otherwise textured to permit a user to rotate it. Rod 120 can be attached to knob 205, such as on an interior surface of knob 205 where there is a break in shaft 215 through which rod 120 may pass. Thus, rod 120 can move longitudinally within elongate curved member 30 as knob 205 is rotated along shaft 215. In yet another embodiment, an actuator can be pivoting, so that as the actuator is pivoted in one direction or the other, rod 120 moves longitudinally within elongate curved member 30. In such an embodiment, the actuator could be operated by an index finger, as with a trigger or a scissors.

In addition to actuator 50, introducer 10 can include other structures for manipulating snare 40 (e.g., for further pulling snare 40 into lumen 105 to more firmly retain the end of a transobturator tape therein). For example, introducer 10 can have an internally threaded knob that is mounted to or extends into second end 65 of handle 20, where it can be engaged with external threads that can be present on the actuator. The knob can be rotated to pull the actuator toward second end 65, thus pulling snare 40 further into elongate curved member 30.

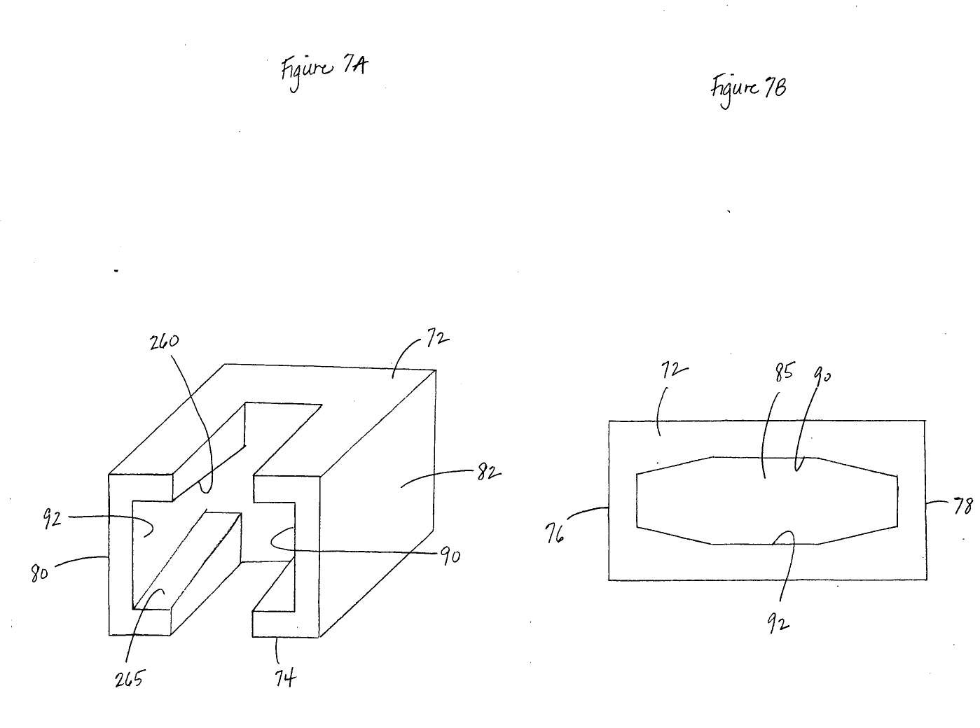

In further embodiments, actuator 50 can have a structure for reversibly locking it into one or more positions (e.g., an extended position or a retracted position). For example, actuator 50 can define one or more indentations or protrusions configured to mate with one or more protrusions or indentations in open portion 85 of handle 20. In one embodiment, first and/or second side surfaces 160 and 165 of actuator 50 can define one or more protrusions that can be configured to engage one or more indentations in first and/or second inner side surfaces 90 and 92. In another embodiment, open portion 85 can be further defined by top inner surface 260 and bottom inner surface 265 of handle 20 as shown in FIG. 7A, for example. Top inner surface 260 and/or bottom inner surface 265 can be sloped such that as actuator 50 approaches first end 60 or second end 65, it becomes reversibly retained by a friction fit. Alternatively, first inner side surface 90 and/or second inner side surface 92 can be angled as shown in FIG. 7B, for example, to achieve a similar friction fit as actuator 50 approaches first end 60 or second end 65. Ratcheting mechanisms also may be provided, and disengagement of the ratchet may “reset” the actuator. Any other suitable mechanism for reversibly holding actuator 50 at one or more positions also can be used.

Actuator 50 can be made using any suitable method, including methods that are standard in the art. For example, actuator 50 can be molded or machined in a single piece or in multiple pieces. In one embodiment, actuator 50 can be molded or machined as two separate pieces that can be assembled and held together using, for example, a snap fit, a friction fit, or a connecting device such as a bolt, a screw, or a clamp. Actuator 50 also can be made from any suitable material. For example, actuator 50 can contain plastic (e.g., a moldable or machinable thermoplastic such as polycarbonate, polypropylene, polyethylene, polystyrene, or polyvinyl chloride, or a thermoplastic copolymer containing, for example, polycarbonate and ABS). Alternatively or in addition, actuator 50 can contain metal (e.g., stainless steel).

Methods

In operation, the introducer devices described herein can be used by a clinician to introduce and position a transobturator tape in a female subject. Methods for using an introducer can include some or all of the following steps:

(1) A midline paraurethral incision can be made in the middle third of the vagina.

(2) One of the two obturator foramina, e.g., the lower internal section, can be located by slipping a finger into the vaginal incision, and an incision can be made in the perineal skin opposite it, i.e., in the groin, so as to form an opening through which the elongate curved member or needle of an introducer device as described herein can be passed. The elongate curved member can be inserted through this skin incision, initially perpendicular to the perineum for about 15 mm (passing through the internal obturator muscle just outside the ischiopubic branch). The elongate member then can be manipulated by the user and allowed to follow its curvature, guided by a finger inserted opposite the obturator muscle through the vaginal incision.

(3) The introducer device can be actuated such that at least the loop at the distal end of the snare is advanced out of the elongate curved member and into the vagina. One end of the tape can be passed through the loop, and the introducer can again be actuated to retract the loop and the end of the tape into the elongate curved member such that the end of the tape is securely retained therein. Threading of the tape may be substantially facilitated by the opening of the loop, when it is extended, into a wider cross-section.

(4) The introducer and the tape can be pulled though the vaginal incision and back to the surface of the skin through the width of the elevator and internal obturator muscles. Once the introducer is withdrawn from the body, the excess tape can be cut off flush with the skin, either before or after the loop is advanced through the distal end of the elongate curved member so that the end of the tape can be removed from the introducer.

(5) The tape then can be placed between the Halban's fascia and the periurethral fasciae to position it such that its central area is located opposite the fasciae. The tape can be positioned without pulling it behind the urethra.

(6) An incision can be made in the perineal skin opposite the second obturator foramen, into which the elongate curved member or needle of the same or a second introducer can be inserted and advanced through the vaginal incision. The loop can be advanced out of the elongate curved member, the free end of the tape can be passed through the loop and retracted into the elongate curved member, and the tape and introducer can be pulled back to the body surface in a similar manner as before.

(7) Once the excess tape is cut off flush with the skin, the skin can be immobilized to disconnect it from the tape. Finally, the incision can be closed (e.g., using a stitch of fast resorbing suture material.)

By positioning the urethral sling in such a manner, the sling essentially forms a V shape within the body, with the point of the V passing under the urethra without changing the position thereof. In addition, a space can be made between, on the one hand, the Halban's fascia, the perineal muscular plane and the anterior insertion of the puborectal muscle and, on the other hand, the periurethral fascias.

The methods described herein can allow for simple fitting of a urethral sling using minimally invasive surgery. Furthermore, they can provide the urethra with firm suspension while at the same time maintaining a certain degree of flexibility and, most of all, keeping the vital organs in the vicinity relatively far away from the sling. Furthermore, these methods can play a part in reconstructing the endopelvic fascia. The methods do not require that all of the described steps be performed. Further, additional or different steps may be included. In addition, the steps need not be performed in the order listed.

In addition to being useful for introducing and positioning a urethral sling within a female subject, the devices described herein also can be used in other procedures to, for example, deliver tissue, sutures, or other materials to the body, or to retrieve such items from the body.

Articles of Manufacture

Also provided herein are articles of manufacture containing one or more (e.g., one, two, three, four, five, six, seven, eight, nine, ten, or more) obturator introducer devices, packaged together with one or more urethral slings (e.g., one or more Mentor ObTape slings). The articles of manufacture also may include a label or insert indicating how the introducer(s) may be used to position a urethral sling within a subject, to deliver other objects to a subject, or to retrieve objects from within a subject. Methods for producing such articles of manufacture are known in the art.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device comprising:
(a) a handle,
(b) an elongate curved member defining a lumen, said member having a proximal end attached to said handle and an open distal end, and
(c) a snare slidably disposed proximate to the distal end of said lumen.

2. The device of claim 1, wherein said snare comprises a loop.

3. The device of claim 2, further comprising a flexible elongate member disposed in said lumen and attached to said loop to move said loop longitudinally in or out of said open distal end.

4. The device of claim 3, wherein said loop expands when extended outside said elongate curved member.

5. The device of claim 2, wherein said loop is comprised of a braided material.

6. The device of claim 1, wherein said snare comprises a pair of interengaging jaws.

7. The device of claim 1, wherein said snare is comprised of metal.

8. The device of claim 7, wherein said metal is nitinol.

9. The device of claim 7, further comprising a coating over said metal.

10. The device of claim 9, wherein said coating is comprised of polyether ether ketone or teflon.

11. The device of claim 1, wherein said snare closes on a material passed through said snare when said snare is retracted at least in part into said open distal end of said elongate curved member.

12. The device of claim 1, wherein said elongate curved member is shaped to be guided in a predetermined path for extending from the vicinity of an obturator foramen in a female subject to the vicinity of the vagina of said subject.

13. The device of claim 12, wherein said predetermined path comprises, from said proximal end toward said distal open end, a first straight segment, a first convex curved segment, a second straight segment, and a second concave curved segment.

14. The device of claim 12, wherein said predetermined path is shaped for positioning a urethral sling between the urethra of a female subject and an obturator foramen of said subject.

15. The device of claim 1, wherein said distal open end comprises a beveled opening.

16. The device of claim 1, wherein said distal open end comprises a flat opening.

17. The device of claim 1, further comprising an actuator associated with said handle for manual operation by a user, wherein said actuator is connected to said snare so that movement of said actuator causes extension or retraction of said snare.

18. The device of claim 17, wherein said actuator is slidable.

19. The device of claim 17, wherein said actuator is pivoting.

20. The device of claim 17, wherein said actuator is rotatable and thereby moves linearly relative to said handle to extend or retract said snare.

21. The device of claim 17, wherein said actuator comprises one or more protrusions to receive force from a user's hand.

22. The device of claim 17, wherein said actuator comprises a top surface and a bottom surface, wherein said top and bottom surfaces define protrusions to receive force from a user's hand.

23. The device of claim 17, wherein said actuator has an slot through which a pin can be passed from a first side of said handle to a second side of said handle.

24. A method for introducing a transobturator into a female subject, said method comprising:
(a) guiding an introducer having a retractable snare through a perineal incision opposite an obturator foramen on a first side of said subject, through the abdomen of said subject, and through a vaginal wall of said subject;
(b) extending said snare from said introducer;
(c) inserting a transobturator into the vagina of said subject and passing a first end of said transobturator through said snare;
(d) retracting said snare to hold said first end of said transobturator; and
(e) withdrawing said transobturator to an area proximate said perineal incision.

25. The method of claim 24, further comprising making an incision in the vaginal wall.

26. The method of claim 24, further comprising withdrawing said first end of said transobturator through said perineal incision.

27. The method of claim 24, further comprising, subsequent to step (e), cutting off an excess portion of said transobturator flush with the skin.

28. The method of claim 24, further comprising repeating steps (a) through (e) on a second side of said subject so that a second end of said transobturator is positioned proximate a perineal incision opposite a second obturator foramen of said subject.

29. A kit comprising a transobturator and one or more transobturator introducers, each of said transobturator introducers comprising a handle, an elongate curved member defining a lumen and having an open distal end and a proximal end attached to said handle, and a snare slidably disposed in said lumen proximate said open distal end.

30. The kit of claim 29, comprising two said transobturator introducers.

31. A device comprising:
(a) a handle,
(b) an elongate curved member defining a lumen, said member having a proximal end attached to said handle and a distal end, and
(c) a means for holding a transobturator near said distal end of said elongate curved member.
